(19) United States
(12) Patent Application Publication
(10) Pub. No.: US 2004/0244073 A1
(43) Pub. Date: Dec. 2, 2004

(54) Process of controlled shuffling of chromosone fragments for plant breeding

(76) Inventors: Victor Klimyuk, Halle/Saale (DE); Serik Eliby, Halle/Saale (DE); Sylvestre Marillonnet, Halle/Saale (DE); Newell Bascomb, Brookside, NJ (US); Yuri Gleba, Halle/Saale (DE)

Correspondence Address: MYERS BIGEL SIBLEY & SAJOVEC, PO BOX 37428, RALEIGH, NC, 27627, US

(21) Appl. No.: 10/481,285

(22) PCT Filed: Jun. 27, 2002
(86) PCT No.: PCT/EP02/07134

(30) Foreign Application Priority Data
Jun. 29, 2001 (DE) 101 31 680.9

(51) Int. Cl. C12N 15/87; A01H 1/00; A01H 5/00; C12N 15/82

(52) U.S. Cl. 800/278; 800/288; 800/260; 800/295; 800/298; 435/468

(57) ABSTRACT

Disclosed is a method of constructing a wild-species genomic library of chromosome fragments incorporated in a crop-species genome. First, a number of transformants for donor and recipient plant species is produced, carrying the DNA constructs necessary for the exchange of chromosomal fragments mediated by site-specific recombination. The donor and recipient are chosen such that, upon sexual cross or somatic cell fusion, they produce unstable progeny or demonstrate preferential segregation or sorting out. Second, the crossing between donor and recipient species and formation of chromosomal recombinants of donor and recipient plant species is induced. Third, taking advantage of the instability of hybrids between donor and recipient, recombinant cells and plants of the recipient are selected which contain specific chromosome fragments of the donor species. Also disclosed are transgenic plants, libraries and breeding material produced by the methods.

### FIELD OF THE INVENTION

The present invention relates to a method of generating plants containing recombined chromosomes and to a library of chromosomal fragments of a plant species with useful traits incorporated into the genome of a distantly related crop plant. Further, it relates to breeding material obtained or obtainable by the method of the invention.

### BACKGROUND OF THE INVENTION

The current gene discovery and functional genomics efforts in agricultural biotechnology companies, life sciences companies and academic laboratories will soon produce a vast number of new and useful genes. Most major genomics efforts so far have emphasized Arabidopsis and a few major crops such as maize, soybean and rice. There are, however, numerous wild species and economically less important species that represent a vast reservoir of useful genetic information that should be explored. The Advanced Backcross method, employing the simultaneous use of molecular markers to identify specific chromosomal regions, has recently been used to identify useful traits in wild relatives of tomato and rice (Tanksley et al., 1996, Teor.Appl Genet., 92:213-224; Yamamoto et al., 2000, Genetics, 154:885-891). To tap into this reservoir, inexpensive gene discovery and/or functional genomics approaches must become available. Development of tools and technological platforms for such limited gene discovery/genomics efforts thus represents an important and urgent task. Such genetic tools and efforts are important because they also could eventually be useful in replacing traditional breeding methods of gene manipulation with more powerful, rapid and precise methods. Current breeding practices, despite the improvement recently made by the use of marker-assisted genotyping (for review see Mazur & Tingey, 1995, Current Opinion in Biotechnology, 6:175-182), still rely on the use of natural uncontrolled chromosome recombination and sexual hybridization processes. Introgression of artificially introduced genes (transgenes) can theoretically be greatly improved and controlled by the use of recombination systems borrowed from microbial or yeast organisms (for review, see Corman & Bullock, 2000, Curr Opin Biotechnol., 11 455-460). However, introgression of useful genes from wild relatives is more difficult as those come as chromosome fragments and the ultimate solutions come either through costly gene identification and isolation (e.g. through positional cloning) or traditional, and very inefficient, recurrent backcrossing (see, e.g., Harlan & Pope, 1922, J. Heredity, 13:319-322).

There are numerous publications and patents describing the use crosses between remote plant species with the purpose of transfering some useful traits from one species to another, for example introgressing Tripsacum germplasm into maize (Maguire, 1962, Can. J. Genet Cytol., 5:414-420; Reeves & Bockholt, 1964, Crop Sci. 4:7-10; deWet et al., 1972, Am. J. Bot., 59:1026-1029; Simone & Hooker, 1976, Proc. Am. Phytopathol., Soc. 3:307; Bergquist, 1981, Phytopathology, 71:518-520; Cohen & Galinat, 1984, Crop Science, 24:1011-1015). Kindiger and Sokolov (U.S. Pat. No. 5,710,367) describe apomictic maize/Tripsacum hybrids useful for introgressing diplosporous apomictic reproduction into a maize background. Galinat (U.S. Pat. No. 4,051,629) describes the use of alien chromosomes from Tripsacum to mask the expression of undesirable recessive traits in the production of hybrid corn seeds. When an alien addition monosome is used as a parent in a hybrid cross, the low transmission rate of the alien chromosome results in the expression of desirable traits in the progeny which were masked with the undesirable traits. Eubanks (US5750828) describes the process of transfer of useful traits' from Tripsacum into maize using intermediate hybrid Tripsacum x Zea diploperennis (Tripsacorn) with better crossability to maize. This partially helps to overcome the difficulties in chromosomal fragment transfer generated by strong cross-incompatibility between maize and Tripsacum chromosomes, which can pair only occasionally (Maguire, M. P., 1961, Evolution, 115:393-400; Maguire, M. P., 1963, Can. J. Genet. Cytol., 5:414-420; Galinat, W. C., 1974, Evolution, 27:644-605). However, all approaches described above produce transfer of chromosomal fragments to occasional (random) sites within the genome of the recipient species, creating complicated patterns of chromosomal rearrangements. Such an approach actually requires a lot of effort each time for achieving a specific goal and does not provide a convenient solution for an efficient manipulation with chromosomal fragments. The most recent patent application by Kuchuk & Klimyuk (WO 0070019) proposes the method of manipulation with transgenes using unstable hybrids. The method described is designed for transgene manipulation, not for the movement of resident chromosomal material between different plant species.

Another approach to transfer desired genetic information between remote plant species is the use of somatic hybridization. Dudits and colleagues (1984, Proc. Natl. Acad. Sci. USA, 84:8434-8438) describe the transfer of resistant traits from carrot into tobacco by asymmetric somatic hybridization. However, the resulting plant material from somatic hybridization experiments is often contaminated with very complex chromosomal rearrangements, which makes this approach of limited use. There is only one publication describing an attempt to use the combination of a site-specific recombination system and somatic hybridization to create hybrid chromosomes between Arabidopsis and tobacco (Koshinsky, H. A., Lee, E. & Ow, D., 2000, Plant J., 23:715-722). This approach produces clearer results compared to those produced by accidental recombination between chromosomes of different species, but no plant tissue carrying an alien chromosome fragment was detected after plant growth without selection. The conclusion was that interspecies transfer of a chromosome arm between plant cells is possible, but maintenance of the hybrid chromosome in a plant is unlikely.

Therefore, it is an object of the invention to provide a novel and highly efficient method for generating transchromosomic plants.

It is another object of the invention to provide a method of creating a library of chromosomal fragments of one plant species which is stably maintained in the genetic background of another plant species.

### SUMMARY OF THE INVENTION

This invention provides a method for generating plants containing recombined chromosomes, said method comprising the following steps:

(a) preparing a donor and a recipient plant both transformed with nucleic acids having sequences that allow for site-specific recombination of said nucleic acids on the chromosomes of the two plants;

(b) crossing the donor and recipient plants to produce progeny;

(c) maintaining said progeny under conditions that allow for elimination of donor chromosomes to take place; and

(d) selecting progeny of said recipient plant which contains chromosomal fragment(s) of said donor plant, said fragment(s) having been acquired by site- specific recombination between said nucleic acids of said donor and said recipient plant.

Exchange of chromosome fragments has the advantage that multigenic traits may be introduced in a recipient plant. This is not possible by conventional processes of producing transgenic plants, wherein a single or a very limited number of transgenes is introduced in a plant by transformation. Furthermore, the genes responsible for a trait do not have to be cloned. These genes do not even have to be known.

This invention allows the exchange of a chromosome fragment between a donor and a recipient plant in a much more controlled and efficient way compared to conventional plant breeding. By the method of the invention, advantageous traits in wild species can be made available to crop plants without knowing the genetics or genome sequence of said wild species.

According to this invention, the donor and the recipient plants have to be endowed with a sequence that allows for site-specific recombination of nucleic acids carrying such sequences. Preferably, many transformants are produced of said donor plant in order to produce a set of transformants having site-specific recombination sites randomly integrated in many different locations and distributed over all chromosomes of said donor plant. This allows sampling of the whole genome and construction of a chromosome fragment library of said donor plant. The size of said library depends directly on the number of transformants of the donor plant, each having a site-specific recombination site at a different location in its genome.

For the recipient plant, a small number of acceptor lines may be sufficient for the method of the invention and for creating said library. This number is in most cases smaller than 10 and preferably only 2 to 3. However, even one may be sufficient. A suitably transformed recipient line has its site-specific recombination site located such that recombination does not remove an essential or otherwise desired function. Suitability of the recipient line may be easily explored.

The nucleic acids used for transformation of said donor and recipient plants may contain one site-specific recombination site. Preferably, they contain two sites for site-specific recombination in different orientations in order to increase the chance of productive recombination. Preferably, site specific recombination can be induced. To this end, either the nucleic acid for the transformation of the donor or that for the transformation of the recipient, or both, may further contain a gene coding for a recombinase functional with said site-specific recombination site under the control of a promoter. Most preferably, the recombinase gene is deleted from the recipient due to the recombination event.

Said nucleic acids may further have a selectable marker for selecting transformants of said donor or said recipient plant.

Further, means for selection of the recombination event are preferably provided (step (d)). This may e.g be achieved by a selection marker gene on one of said nucleic acids for the transformation of said donor or said recipient plant. More preferably, said selection marker becomes active as a result of site-specific recombination e.g. by assembling a full expression cassette or a functional coding sequence of the marker gene. As an example of this important embodiment, the nucleic acid shown in FIG. 3 may be used for transforming the donor plant(s) and that shown in FIG. 7 may be used for transforming the recipient plant(s). In this case, the BAR gene is placed under the control of a promoter upon recombination and the recombinase gene is removed from the recipient.

Further, said nucleic acids having sequences that allow for site-specific recombination may comprise transposon element(s), whereby the transposon element(s) can be used for mapping resultant chromosome recombinants by insertional mutagenesis (e.g. transposon tagging, transposon mutagenesis). Preferably, said transposon element(s) are inactive, i.e. they lack a suitable transposase. Transposition may then be activated by providing a transposase functional with said transposon. This may e.g. be accomplished by crossing the plant with another plant expressing the transposase.

The process of the invention may be applied to donor/recipient pairs that are closely or distantly related. Preferably, the donor and the recipient are selected such that progeny (cells) obtained upon crossing form unstable hybrids for allowing chromosomes of the donor to get preferentially lost. Further, the duration of coexistance of chromosomes of the donor and the recipient in the progeny produced should be sufficient for site-specific recombination. These criteria may be tested experimentally for (see further below).

The transformed donor and recipient plants may be crossed sexually or non-sexually, i.e. by somatic cell fusion. Sexual crossing may be used if the donor and recipient plants are related sufficiently close for sexual crossing. This will generally be the case if they belong to the same taxonomic family, preferably to the same genus. Crossing by somatic cell fusion, allows the application of the method of the invention to donor and recipient plants which are too distantly related for sexual crossing or when chromosome mixing is unlikely. In this way, chromosome fragments may be exchanged between plants belonging to different families or to different classes. As an example, hybrids of monocots and dicots may be created by somatic cell fusion. If desired, somatic cell fusion may of course also be used in the case of close relationship. The conditions required for elimination of donor chromosomes in the progeny after somatic cell fusion are known in the art.

After crossing said donor and recipient plants to produce progeny, said progeny is maintained under conditions that allow for elimination of donor chromosomes to take place. This may happen by chromosome segregation (sorting out). Preferably, said progeny is unstable such that chromosomes of the donor are preferentially lost. The higher the unstability of the progeny, the faster unwanted chromosomes of the donor (chromosomes not endowed with said nucleic acid for site-specific recombination) get lost. Preferably, said elimination of donor chromosomes can be achieved in a small number of generations, most preferably in 1 or 2 generations. The duration of coexistance of chromosomes of the donor and the recipient in the progeny produced should be sufficient for site-specific recombination.

Examples of donor/recipient pairs include Tripsacum/barley, Tripsacum/oat, Orychophragmus/a crucifer (canola or rape seed), Glycine tomentella/soybean, Solanum phreja/potato, maize/wheat, maize/barley, maize/oats, Pennisetum/wheat, Pennisetum/barley, Pennisetum/maize, Hordeum bulbosum/barley, Hordeum bulbosum/wheat, Oryza minuta/rice, Tripsacum/wheat, Tripsacum/maize, Nicotiana africana/Nicotiana tabacum. Further, said donor or said recipient plant may be soybean carrying a ms mutation causing polyembryony or one or both of said donor and recipient plants may be cotton carrying a Se semigamy mutation.

This invention further relates to a library of chromosomal fragments of one or more heterologous species incorporated into a genome of another species, preferably a crop species, obtained or obtainable by the method of this invention. Such a library, which may be in the form of seeds, allows to select a transchromosomic recipient plant having a desired trait from another species. The library may contain any trait of the donor plant. A transchromosomic recipient plant expressing a useful trait may then be selected from said library by observing a certain phenotype (e.g. by comparing protein expression profiles to the starting recipient plant, which may be done by 2-D gel electrophoresis in combination with mass spectroscopy) or by genetic analysis. Members of said library may e.g. be used in functional genomics studies and as a starting breeding material.

The invention also comprises plants, plant material, and breeding material of crop species, obtained or obtainable by the method of the invention or products derived therefrom.

### BRIEF DESCRIPTION OF THE DRAWINGS

FIG. 1 shows a general principle of shuffling the chromosomal fragments between donor and acceptor plant species; the star indicates a transposon. The recipient plant and the result of the first crossing contain an inactive transposon which becomes active upon crossing with a plant functioning as a transposase source providing a transposase.

FIG. 2 is a linear plasmid map of pIC1600;

FIG. 3 is a linear plasmid map of pIC3714;

FIG. 4 is a linear plasmid map of pIC529;

FIG. 5 is a linear plasmid map of pIC1251;

FIG. 6 is a linear plasmid map of pIC1262;

FIG. 7 is a linear plasmid map of pIC4041.

Appendices 1 to depict constructs used in the examples.

### DETAILED DESCRIPTION OF THE INVENTION

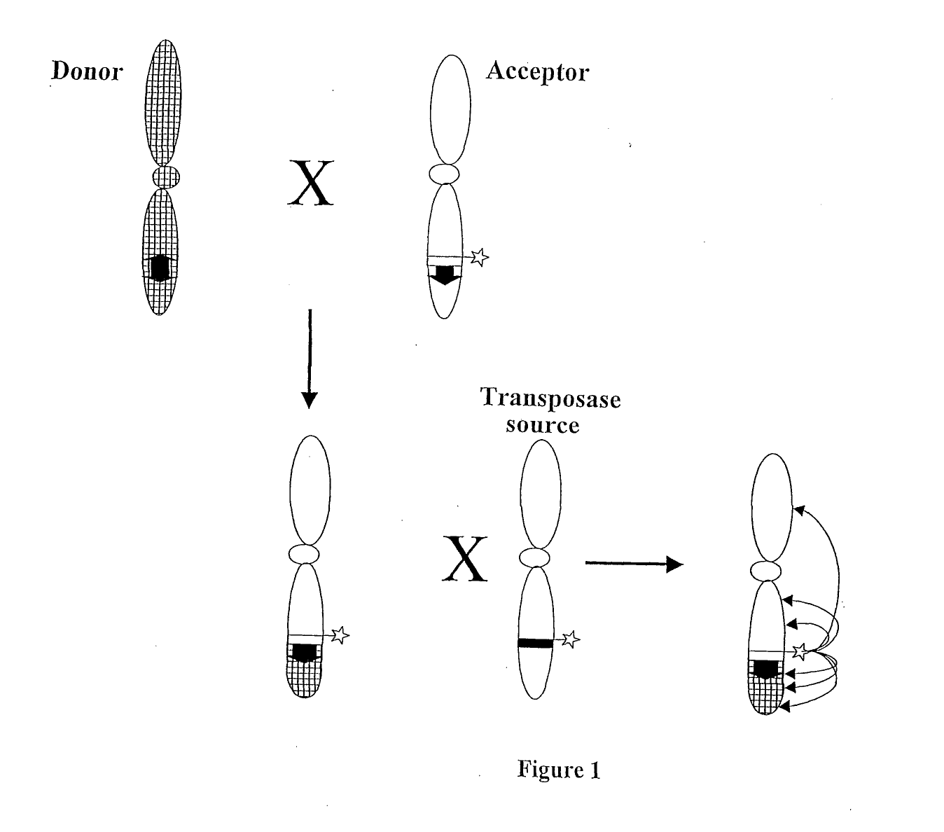

The approach described in this invention relies on use of a combination of several technologies that have been used previously, but for different purposes. This approach represents a novel way of constructing a full wild-species genome library of chromosome fragments in the genome of a crop-species recipient. The general principle of the approach is shown in FIG. 1. First, a number of transformants of the donor is preferably generated having insertions of recombination sites evenly distributed among all chromosomes. The acceptor (recipient) plant requires the generation of a smaller number of insertions of recombination sites. However, the transgenic recipient plants are preferably tested on the tolerance of a deletion of a distal part of a recipient chromosome following substitution with a random heterologous chromosomal fragment. Theoretically, 2-3 tested acceptor lines may be sufficient for creating a library.

The second step includes performing crosses between the donor and acceptor plant species and preferably selecting for the progeny of the acceptor plant carrying a chromosome fragment of the donor plant. Finally, a more detailed identification and isolation may be accomplished by gene tagging in a line with a specific chromosome fragment.

This invention deals preferably with wheat as a crop species of interest and maize and pearl millet as donors of useful trait(s). In addition to the development of a novel genomics and breeding platform, this approach can target the introgression of useful traits (apomixis, pathogen resistance, etc) into wheat germplasm. The approach of this invention may be applied to any crop species of interest which can form unstable hybrids with the donor plant carrying useful traits (Tripsacum/maize; Orychophragmus/Brassica, Glycine tomentella/soybean, Solanum phureja/Solanum tuberosum, etc.). In general, for every crop species (including all varieties and lines thereof), there is a wild relative or a mutant form, that, upon hybridization, forms an unstable hybrid and can serve as a donor or clipboard plant as defined herein. An empirical way of identifying such an organism involves crossing a crop species of interest with a number of related species and testing the genetic makeup of the resulting progeny. Methods of preliminary identification of progeny that is predominantly uniparental are known in the literature and are based on different selective or non-selective traits. Methods of broad and reliable genotyping of the progeny are numerous, simple, and rely on the analysis of various markers in genomic DNA. Based on such primary screening and subsequent genotyping, suitable clipboard organisms can be rapidly identified. Beyond this basic criterion, the donor/recipient pairs are preferably chosen so as to provide an adequate duration of the hybrid state in cells of a primary hybrid or of its progeny. While complete elimination is a desired end state, the relative duration of the coexistence of chromosomes of both species in the same cell is important as it provides sufficient time for the exchange of chromosomal fragments between the donor and acceptor plant species.

The present invention is preferably directed to a method for introducing genetic material as chromosomal fragments into plants, comprising:

preparing a donor plant transformed with a heterologous nucleic acid having sequence(s) recognized by a site-specific recombinase and a promoterless selectable marker that allows recombination between alien chromosomes carrying such sequences and selection for recombination events;

preparing a recipient plant transformed with a heterologous nucleic acid having sequence(s) recognized by a site-specific recombinase and the gene encoding for such recombinase under the control of an inducible, tissue-specific or constitutive promoter;

crossing the recipient plant and the donor plant, wherein the donor and recipient plants, upon crossing, produce unstable progeny or demonstrate preferential segregation or sorting out;

and selecting progeny of the recipient plant, which contain a chromosomal fragment of the donor in the genome.

The methods of the present invention provide for chromosome fragment manipulation in essentially all crop species, especially the economically important varieties.

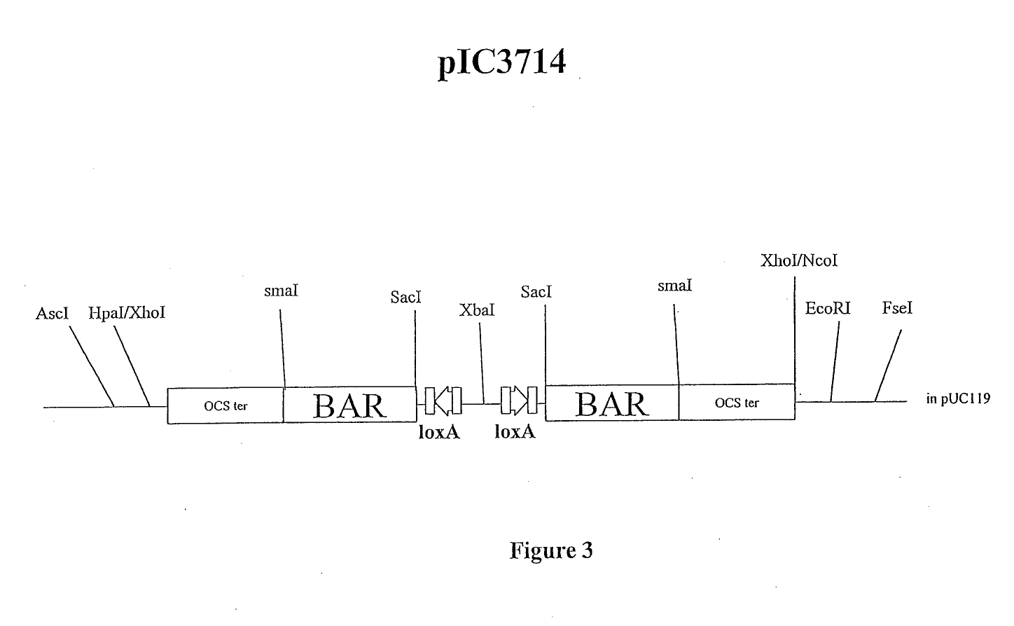

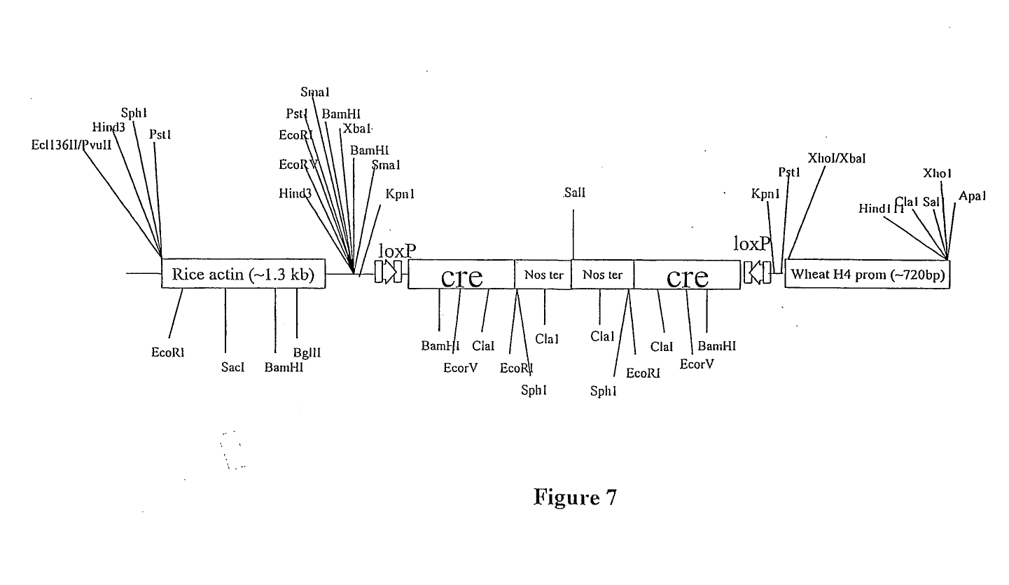

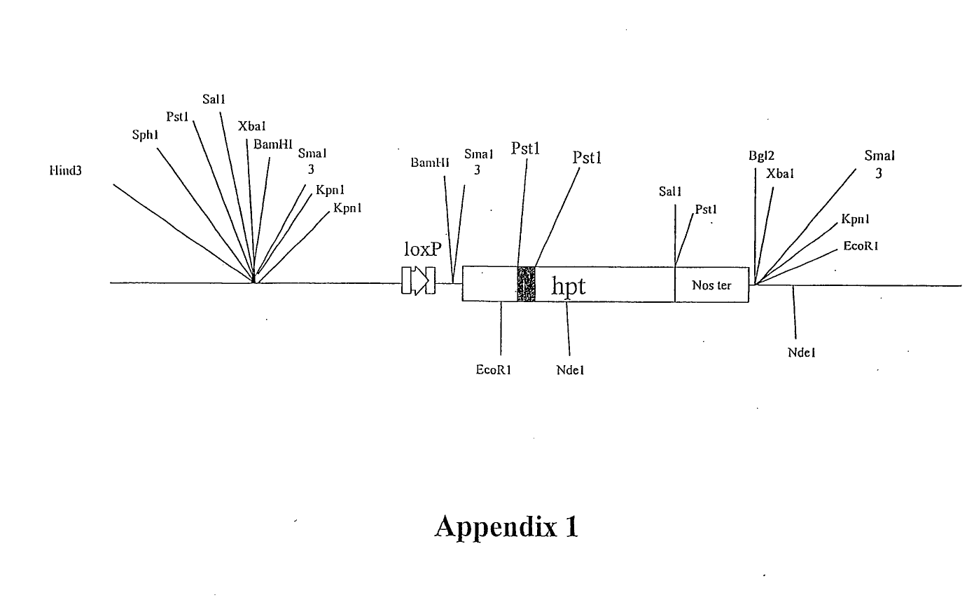

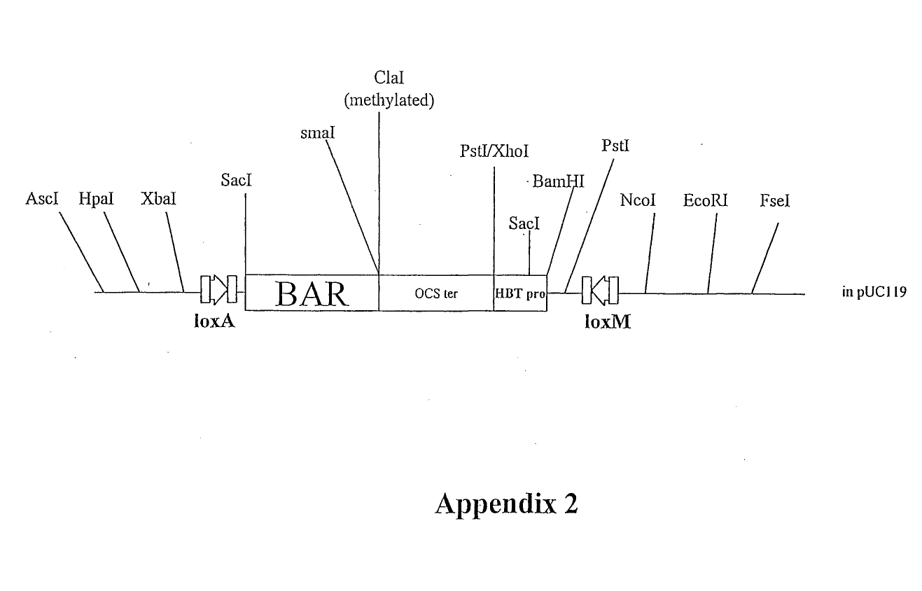

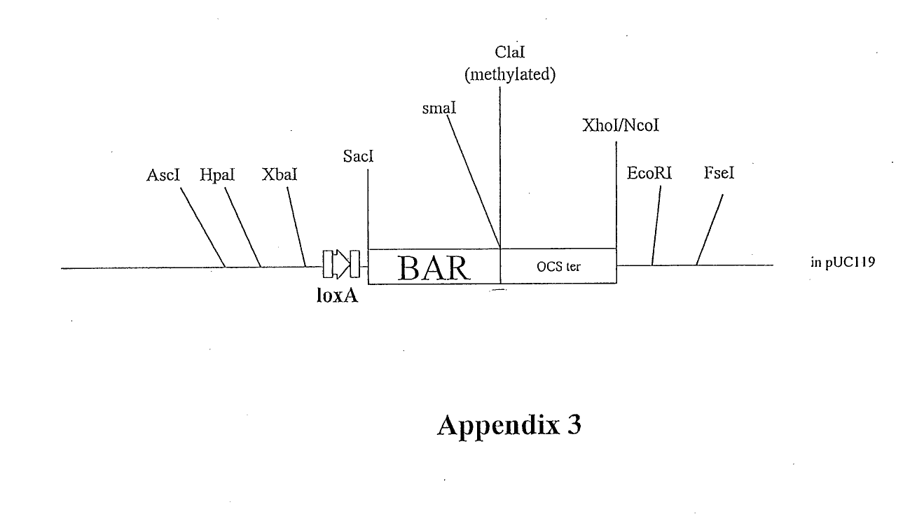

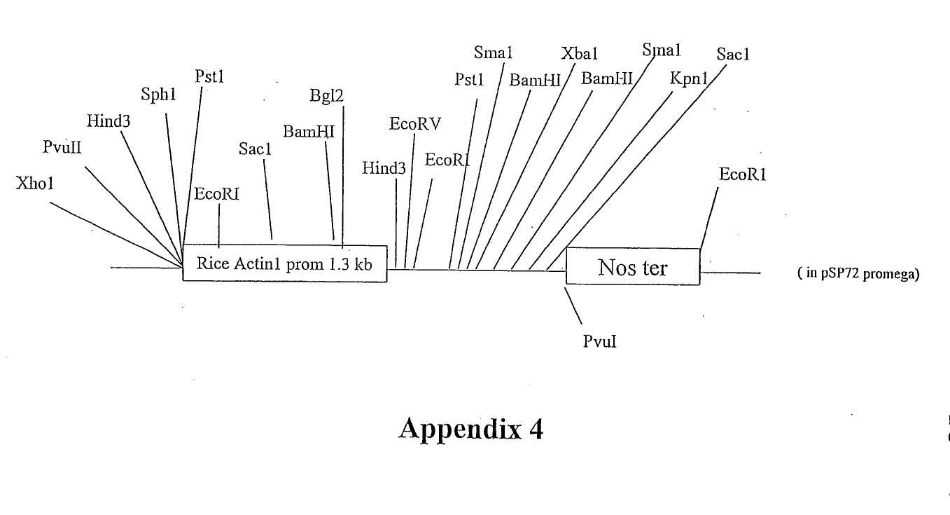

The orientation of recombination sites in the donor and acceptor plants toward the centromeres preferably coincide in order to provide successful recombination events. If the recombination sites are differently oriented, the recombination events will lead to the formation of dicentric chromosomes which are unstable. To address this problem and in order to increase the efficiency of the system, the constructs for transforming the donor (e.g. pIC3714, FIG. 3) and one of the constructs for acceptor (e.g. pIC4041, FIG. 7) plant species preferably have recombination sites in both orientations. Recombination events between the plant chromosomes carrying such constructs always have a good chance to produce a recombinant chromosome without the recombinase, but with a selectable marker being placed under the control of promoter.

In a preferred embodiment of the invention, the recombination of chromosomal fragments is caused by the use of a recombinase under the control of, for example, either the rice actin or wheat histone H4 promoter. In another embodiment of the invention, two different promoters may be used simultaneously to drive the expression of the recombinase gene (see FIG. 7), thus increasing the probability of recombination events during the short period of coexistence of alien genomes.

Site-specific recombinases from bacteriophage and yeasts are widely used as tools for manipulating DNA both in the test-tube and in living organisms. Preferred recombinases/recombination site combinations for use in the present invention are Cre-Lox, FLP-FRT, and R-RS, where Cre, FLP and R are recombinases, and Lox, FRT, and RS are the recombination sites. Other suitable systems include the intron-encoded yeast endonuclease I-SceI, see Choulika, et al., Mol. Cell Biol. 15:1968-1973 (1995). To be functional in plants, these sites require 7-8 base pairs (bp) of core sequence between 12-13 bp inverted repeats; the asymmetric core site determines the site orientation, and thus the types of recombination product. Regardless of whether recombination sites are placed on or within a single DNA molecule in direct or opposite orientation, or are placed on unlinked linear or circular DNA molecules, the corresponding recombinase can catalyze the reciprocal exchange to produce a deletion, inversion, translocation or co-integration event. See, Bollag, et al., Ann. Rev. Genet. 23:199-225 (1989); Kilby, et al., Trends Genet. 9:413-421 (1993); and Ow, Curr. Opinion Biotech. 7:181-186 (1996). In the present invention, recombinase-mediated site-specific translocation occurs between different, and in particular non-homologous chromosomes. This in trans recombinase effect is essential in order to effect transfer of chromosomal fragments between two chromosomes belonging to different parents in a hybrid. See, Koshinsky et al., 2000, Plant J., 23:715-722.

Examples of suitable homologous recombination systems for the use in the present invention are disclosed in the literature and include inter alia the Cre-Lox system (Sauer, U.S. Pat. No. 4,959,317, Odell, et al., U.S. Pat. No. 5,658,772; Odell, et al., PCT WO91/09957) and the FLP-FRT system (Hodges and Lyznik, U.S. Pat. No. 5,527,695). One particular utility of known recombination systems for transgene management in plants is directed excision of a transgene from a plant genome, a procedure that allows elimination of unwanted heterologous genetic material such as antibiotic selective markers from a commercial variety (Ow and Dale, PCT WO93/01283), These systems, however, address an entirely different utility area, namely, the use of homologous recombination to eliminate unwanted portions of heterologous DNA, rather than to manage the flow of heterologous chromosomal fragments.

Homologous recombination-based chromosomal fragment shuffling has both clear and strong advantages. By employing precise targeting via homology-addressed DNA sites, chromosomal fragment “landing sites” can be created that are carefully selected and characterized in advance. As a result, a higher level of predictability and reproducibility of heterologous chromosomal fragment behavior, including heritability, can be achieved.

In a specific embodiment, a transposable element can be used in one of the constructs, so that it will be incorporated into the recombinant chromosome for the purposes of insertional mutagenesis in a heterologous chromosomal region. The tendency of transposable elements to transpose to closely linked sites is a well established phenomenon (Jones et al., 1990, Plant Cell, 8:701-707; Osborne et al., 1991, Genetics, 129:833-844; Carroll et al., 1995, Genetics, 139:407-420). This can be a useful tool for the identification and isolation of useful genes from a heterologous chromosomal region. Preferably, the transposable element is non-autonomous requiring a source of a transposase for its transposition. A transposase may be provided in trans when necessary by crossing with a plant carrying a stabilized transposase gene (see FIG. 1). Plant transposons are among the first mobile DNA elements described and a number of plant transposable elements that have been cloned, such as Ac/Ds, Mu and En/Spm, may be used in the present invention. These transposable elements are currently used as genetic tools in plant molecular biology and biotechnology. They serve as invaluable tools for plant developmental studies, for plant genome analysis and plant gene isolation by so-called insertional mutagenesis and tagging. See, e.g., Walbot, Ann. Rev. Plant Mol. Biol. 43:49-82 (1992). Other examples of transposable elements which may be used in this invention are described in Fedoroff, U.S. Pat. No. 4,732,856; Doonerk et al., PCT Application WO91/156074; etc.), Yoder and Lassner, PCT Application WO92/01370, and Ebinuma et al., PCT Application WO96/15252.

The heterologous DNA may be introduced into the donor and acceptor plants in accordance with standard techniques. Transformation techniques for dicotyledons are well known in the art and include Agrobacterium-based techniques and techniques which do not require Agrobacterium. Non-Agrobacterium techniques involve the uptake of exogenous genetic material directly by protoplasts or cells. These techniques include PEG or electroporation mediated uptake, particle bombardment-mediated delivery and microinjection. Examples of these techniques are described in Paszkowski et al., EMBO J. 3:2717-2722 (1984), Potrykus et al., Mol. Gen. Genet. 199:169-177 (1985), Reich et al., Biotechnology 4:1001-1004 (1986), and Klein et al., Nature 327:70-73 (1987). In each case, the transformed cells are regenerated to whole plants using standard techniques.

Agrobacterium-mediated transformation is a preferred technique for the transformation of dicotyledons because of its high transformation efficiency and its broad utility with many different species. The many crop species which are routinely transformable by Agrobacterium include tobacco, tomato, sunflower, cotton, oilseed rape, potato, soybean, alfalfa and poplar (EP 0 317 511 (cotton), EP 0 249 432 (tomato), WO 87/07299 (Brassica), U.S. Pat. No. 4,795,855 (poplar)). Agrobacterium transformation typically involves the transfer of the binary vector carrying the foreign DNA of interest (e.g,. pCIB200 or pCIB2001) to an appropriate Agrobacterium strain which may depend on the complement of vir genes carried by the host Agrobacterium strain either on a co-resident plasmid or chromosomally (e.g., strain CIB542 for pCIB200 (Uknes et al., Plant Cell 5:159-169 (1993)). The transfer of the recombinant binary vector, to Agrobacterium is accomplished by a triparental mating procedure using E. coli carrying the recombinant binary vector, a helper E. coli strain which carries a plasmid such as pRK2013 which is able to mobilize the recombinant binary vector to the target Agrobacterium strain. Alternatively, the recombinant binary vector is transferred to Agrobacterium by DNA transformation (Höfgen & Willmitzer, Nucl. Acids Res. 16, 9877 (1988)).

Transformation of the target plant species by recombinant Agrobacterium usually involves co-cultivation of the Agrobacterium with explants from the plant following protocols known in the art. Transformed tissue carrying an antibiotic or herbicide resistance marker present between the binary plasmid T-DNA borders is regenerated on selectable medium.

Preferred transformation techniques for monocots include direct gene transfer into protoplasts using PEG or electroporation techniques and particle bombardment into callus tissue. Transformation can be undertaken with a single DNA species or multiple DNA species (i.e. co-transformation) and both these techniques are suitable for use with this invention. Co-transformation may have the advantage of avoiding complex vector construction and of generating transgenic plants with unlinked loci for the gene of interest and the selectable marker, enabling the removal of the selectable marker in subsequent generations, should this be regarded desirable. However, a disadvantage of the use of co-transformation is the less than 100% frequency with which separate DNA species are integrated into the genome (Schocher et al/, Biotechnology 4:1093-1096 (1986)).

Published Patent Applications EP 0 292 435, EP 0 392 225 and WO 93/07278 describe techniques for the preparation of callus and protoplasts of maize, transformation of protoplasts using PEG or electroporation, and the regeneration of maize plants from transformed protoplasts. Gordon-Kamm, et al., Plant Cell 2:603-618 (1990), and Fromm, et al., Biotechnology 11:194-200 (1993), describe techniques for the transformation of elite inbred lines of maize by particle bombardment.

Transformation of rice can also be undertaken by direct gene transfer techniques utilizing protoplasts or particle bombardment. Protoplast-mediated transformation has been described for Japonica-types and Indica-types (Zhange, et al., Plant Cell Rep. 7:739-384 (1988); Shimamoto, et al., Nature 338:274-277 (1989); Datta, et al., Biotechnology 8:736-740 (1990)). Both types are also routinely transformable using particle bombardment (Christou, et al., Biotechnology 9:957-962 (1991)).

Patent Application EP 0 332 581 describes techniques for the generation, transformation and regeneration of Pooideae protoplasts. Furthermore, wheat transformation is described in Vasil, et al., Biotechnology 10:667-674 (1992) using particle bombardment into cells of type C long-term regenerable callus, Vasil, et al., Biotechnology 11:1553-1558 (1993) and Weeks, et al., Plant Physiol. 102:1077-1084 (1993) describe particle bombardment of immature embryos and immature embryo-derived callus.

Transformation of monocot cells such as Zea mays is achieved by bringing the monocot cells into contact with a multiplicity of needle-like bodies on which these cells may be impaled, causing a rupture in the cell wall thereby allowing entry of transforming DNA into the cells (ee U.S. Pat. No. 5,302,523). Transformation techniques applicable to both monocots and dicots are also disclosed in the following U.S. Pat. No. 5,240,855 (particle gun); U.S. Pat. No. 5,204,253 (cold gas shock accelerated microprojectiles); U.S. Pat. No. 5,179,022 (biolistic apparatus); 4,743,548 and U.S. Pat. No. 5,114,854 (microinjection); and U.S. Pat. Nos. 5,149,655 5,120,657 (accelerated particle mediated transformation); U.S. Pat. No. 5,066,587 (gas driven microprojectile accelerator); U.S. Pat. No. 5,015,580 (particle-mediated transformation of soy bean plants); U.S. Pat. No. 5,013,660 (laser beam-mediated transformation); U.S. Pat. Nos. 4,849,355 and 4,663,292.

Plant cells or plant tissue transformed by one of the methods described above are then grown into full plants in accordance with standard techniques. Transgenic seeds can be obtained from transgenic flowering plants in accordance with standard techniques. Likewise, non-flowering plants such as potato and sugar beets can be propagated by a variety of known procedures. See, e.g., Newell et al. Plant Cell Rep. 10:30-34 (1991) (disclosing potato transformation by stem culture).

The examples presented below are summaries of successful transformation and chromosomal fragments transfer for two combinations of important crop species (Pennisetum/wheat and maize/wheat) based on the use of site-specific recombinases. These examples are presented merely to illustrate specific embodiments of the present invention, and are not intended to provide any limitations to the invention not set forth in the claims.

### EXAMPLES

### Reference Example I

Designing the Constructs for Shuffling Chromosomal Fragments

The vectors were constructed using standard molecular biology techniques (Maniatis et al., 1982, Molecular cloning: a Laboratory Manual. Cold Spring Harbor Laboratory, New York).

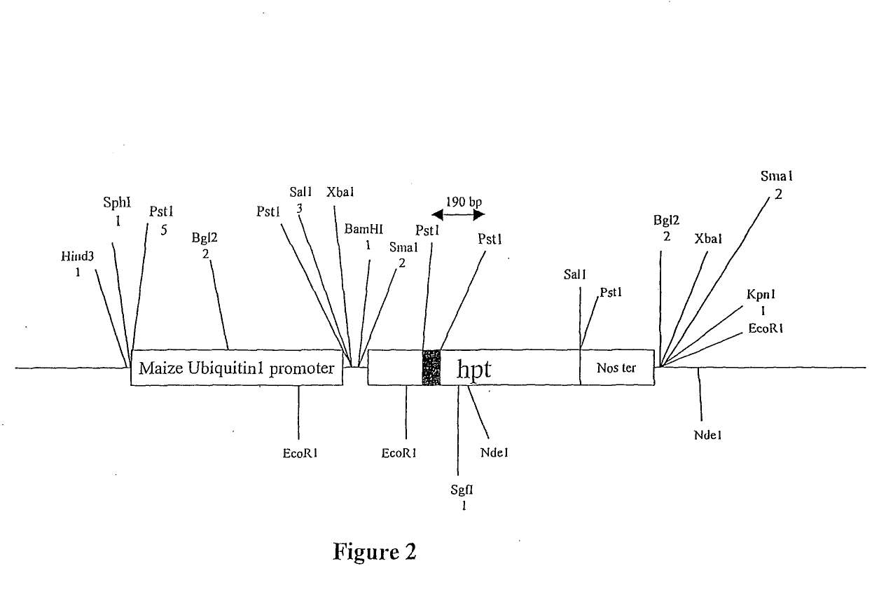

Plasmid pIC1600 (FIG. 2) was generated by simple replacement of small HindIII-BamHI fragment of pIC1551(Appendix 1) with the HindIII-BamHI fragment containing the maize ubiquitin promoter. Vector pIC3714 (FIG. 3) was created as follows.

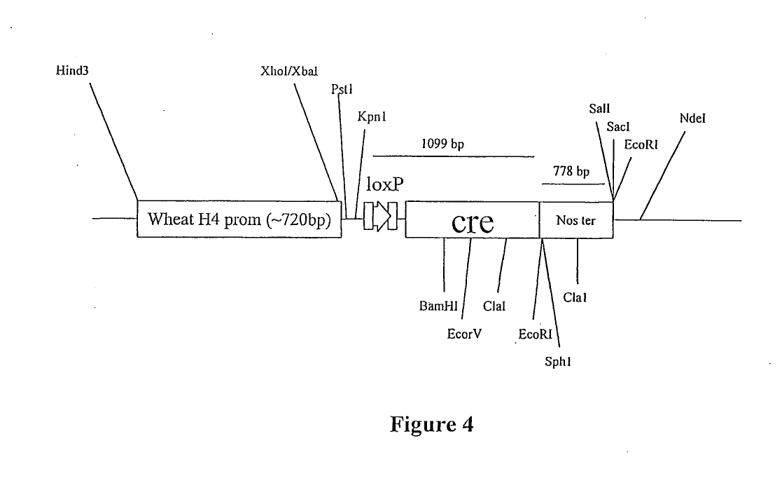

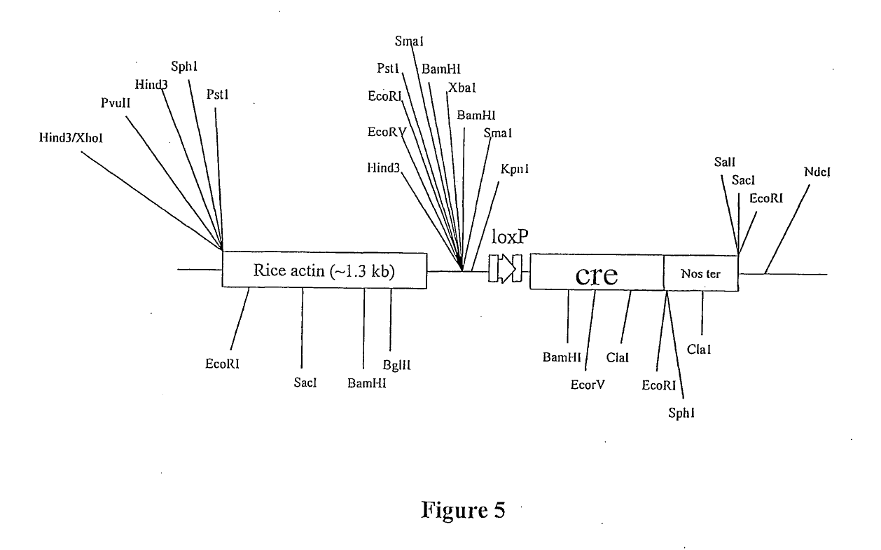

The plasmid pIC607 (Appendix 2) was digested with Xho1 and Nco1, blunt-ended by treatment with Klenow fragment of DNA polymerase I, and the gel purified large fragment was self-ligated yielding the plasmid pIC3709 (Appendix 3). The large Xba1-Hpa1 fragment of pIC3709 was ligated with small Xba1-Xho1/Klenow fragment of pIC607 producing pIC3714. Plasmid pIC529 (FIG. 4) was created by replacing 35S promoter of pIC08 (HindIII-XbaI/Klenow fragment) with HindIII-XhoI fragment of pIC09 containing wheat histone H4 promoter (Tabata et al., 1984, Gene, 31:285-289). Plasmid pIC1251 (FIG. 5) was made by ligation of the large HindIII/Klenow-KpnI fragment of pIC529 with 1.3 Kb XhoI/Klenow-KpnI fragment of pIC044 (Appendix 4), replacing the wheat histone H4 promoter with the rice actin one (McElroy et al., 1990, Plant Cell, 2:163-171).

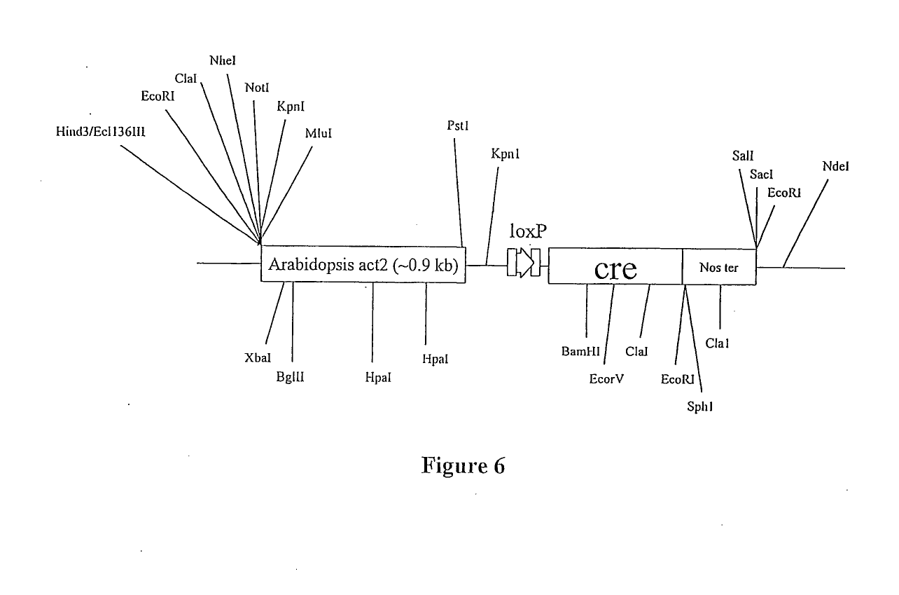

Ligation of HindIII/Klenow-PstI fragment of pIC529 with 0.9 Kb Ecl136II-Pst1 fragment of pIC04 gives plasmid pIC1262 (FIG. 6) carrying the cre recombinase under control of Arabidopsis actin 2 promoter (An et al., 1996, Plant J.,10:107-121).

Plasmid pIC4041 (FIG. 7) was made by co-ligation of 2.6 kb HindIII-Sal1 fragment of pIC529, 3.2 KbPvuII-Sal1 fragment of pIC1251 and gel-purified vector pBS(KS+), treated with HindIII and Ecl13611.

### Example 1

Transformation of Wheat and Pennicetum.

Wheat immature embryo cultures (cvs Bobwhite and Chinese Spring) and maize (cv Hi II) were co-bombarded by pIC1251 (FIG. 5) and pIC1600 (FIG. 2). The results of some transformation experiments for wheat and millet are shown in the table below.

| | | Embryos | Transformants, | Transformants |
|---|---|---|---|---|
| Experiment | Cultivar | shot | with pIC1600 | with pIC1251 |
| 1 | Bobwhite | 200 | 5 | 3 |
| | Chinese | 200 | 4 | 2 |
| | Spring | | | |
| 2 | Bobwhite | 800 | 28 | 15 |
| | Chinese | 200 | 3 | 1 |
| | Spring | | | |
| 3 | Bobwhite | 400 | 9 | 6 |

Millet cultures were co-bombarded by pIC1600 and pIC3714 (FIG. 3):

| | | Embryos | Transformants, | Transformants |
|---|---|---|---|---|
| Experiment | Line | shot | with pIC1600 | with pIC3714 |
| 1 | PEN3 | 300 | 17 | 9 |
| | HGM100 | 200 | 2 | 2 |
| 2 | PEN3 | 400 | 13 | 8 |
| | HGM100 | 300 | 9 | 6 |

Additional co-bombardments of wheat were performed with pIC1600 and pIC529.

Callus Culture Maintenance

Immature seeds of wheat cvs. Chinese Spring and Bobwhite, millet lines PEN3 and HGM100 and maize Hi II were surface-sterilized by immersing into 70% ethanol for 2 min, followed by incubation in 1% Sodium Hypochlorite solution with shaking at 125 rpm for 20 min and finally by 5 washes in sterile distilled water. Immature embryos (1.0-1.5 mm in length, semitransparent) were isolated aseptically and were placed, with a scutellum side up, on an appropriate culture medium solidified by 0.25% phytagel. Embryos developing compact nodular calli were selected using stereomicroscope and used for bombardment 5- 10 days after isolation. The cultures were kept in the dark at 25° C.

Wheat and millet calli were kept on solid MS (Murashige and Skoog 1962) with 2 mg/l 2,4-D (MS2).

Plasmid Preparation and DNA-Gold Coating

The plasmid constructs pIC1251, pIC1600, pIC529, pIC3714 and pIC4041 were purified using Qiagen kits.

A DNA-gold coating according to the original Bio-Rad's protocol (Spd/Ca, double aliquots; Sanford et al., 1993) was done as follows: 50 ml of gold powder (1.0 μm) in 50% glycerol was mixed with 10 μl DNA (1 μg/μl), 50 μl CaCl₂ (2.5M) and 20 μl of 0.1 M spermidine. For co-transformation the plasmids were mixed at a ratio 1:1 (5 μg+5 μg). The mixture was vortexed for 2 min, followed by incubation for 30 min at room temperature, brief centrifugation, washing by 70% and 99.5% ethanol. Finally, pellet was resuspended in 60 μl of 99.5% ethanol (6 μl/shot). All manipulations were done at room temperature.

Bombardment

Microprojectile bombardment was performed utilizing the Biolistic PDS-1000/He Particle Delivery System (Bio-Rad). Immature embryos were pretreated for 4 hours on MS2 medium supplemented by 0.2M of mannitol and 0.2M of sorbitol. Embryos (50/plate) placed in the center of the plate to form a circle with a diameter of 10 mm were bombarded at 1100 psi, with the 15 mm distance from a macrocarrier launch point to the stopping screen and 60 mm distance from the stopping screen to a target tissue. The distance between rupture disk and launch point of the macrocarrier was 12 mm. The calli were transferred to MS2 medium 16 hours after treatment and grown in dark for one week.

Selection, Regeneration

Two days after bombardment the treated calli were transferred to MS selection medium with 2.0 mg/l 2,4-D and 150 mg/l hygromycin B and cultured on light. Four weeks later, greening callus tissues were subcultured to the MS regeneration medium supplemented with 1 mg/l BAP, 0.5 mg/l kinetin, 0.01 mg/l 2,4-D and 150 mg/l hygromycin B. Regenerating plantlets were transferred to jars with the half-strength hormone-free MS medium with 100 mg/l hygromycin B. The fully developed plantlets were acclimated for 7-10 days at 15° C. in a liquid medium containing the four-fold diluted MS salts. Plants with strong roots were transplanted into soil and grown under greenhouse conditions to maturity.

DNA Isolation

Leaf samples were homogenized in Eppendorf tubes with a sand powder in 0.3-5.0 ml of hot (55° C.) 2×CTAB solution. Equal volume of the CTAB solution and 0.6-10.0 ml of Chloroform-Isoamyl alcohol mixture (24:1 v/v) were added to the extracts. The tubes were incubated on a shaker (Mild Mixer PR-12, TAITEC) at a speed 5 at room temperature for 15-30 min. Phases were separated by centrifugation (3600-15 000 rpm, 20° C., 5 min) and the supernatants were carefully transferred into new tubes with 0.6-10.0 ml isopropanol. DNA pellets (12000-15000 rpm, 20° C., 20 min) were washed by 70% ethanol, resuspended in 0.3-1.0 ml TE and RNAse treated for 30 min. After two sequential chloroform extractions DNA samples were pelleted by adding 0.1-0.33 ml of 10 M NH₄Ac and 0.3-1.0 ml of isopropanol (15 000 rpm, 20° C., 20 min). Pellets were washed by 70% and 99.5% ethanol and redissolved in 20-500 μl of 0.1 TE.

PCR Analysis and Southern Hybridization

Primers specific for bar and hpt genes were ordered from Gibco-BRL:

bar primers:
5′-ACCATCGTCAACCACTACATCGAG,
5′-AGGCTGAAGTCCAGCTGCCAGA
hpt primers:
5′-AGACCTGCCTGAAACCGAACTGC,
5′-CACGGCCTCCAGAAGAAGATGTTG

Internal fragments of a bar coding sequence of 442 bp and a hpt coding sequence of 600 bp in length were amplified under the following PCR conditions:

95° C. 2 min; 94° C. 1 min, 62° C. (0.5° C. down/cycle) 1 min, 72° C. 1 min 10 cycles,

94° C. 1 min, 57° C. 1 min, 72° C. 1 min (4 sec up/cycle) 35 cycles.

Aliquotes of 0.5-1 μg of DNA per sample (100 μl) were used in PCR reactions.

Southern Hybridization was performed according to the protocol of the ECL direct nucleic acid labelling system (Amersham, UK).

### Example 2

Generation of Pennicetum Chromosomal Fragments Library on the Genetic Background of Wheat.

The crosses between Pennicetum and wheat were performed as described in Riera-Lizararu & Mujeeb-Kazi, Crop Sci. 33:973-976 (1993). Primary converted lines were selected as F_{O} diploidized haploids emerging from the crosses. The wheat lines carrying chromosomal fragments of Pennisetum were easily selected due to their BASTA resistance. The recombination between Lox sites of pIC3714 and, as a matyter of choice, of pIC529, pIC1251 or pIC4041 resulted in BAR gene activation due to its placement under the control of either the wheat histone H4 or the rice actin promoter.

### Example 3

Generation of Maize Chromosome Fragments Library on the Genetic Background of Wheat.

The crosses between maize and wheat were performed as described by Matzk & Mahn (1994, Plant Breeding, 1 13:125-129).

The wheat lines carrying chromosomal fragments of maize transferred through site- specific recombination were selected as described in example 2.

1. A method for generating plants containing recombined chromosomes, said method comprising the following steps:
(a) preparing a donor and a recipient plant both transformed with nucleic acids having sequences that allow for site-specific recombination of said nucleic acids on the chromosomes of the two plants;
(b) crossing the donor and recipient plants to produce progeny;
(c) maintaining said progeny under conditions that allow for elimination of donor chromosomes to take place; and
(d) selecting progeny of said recipient plant which contains chromosomal fragment(s) of said donor plant, said fragment(s) having been acquired by site specific recombination between said nucleic acids of said donor and said recipient plant.

2. The method of claim 1, wherein said site-specific recombination is induced.

3. The method of claim 1, wherein said sequences are recombination sites and wherein said donor plant, said recipient plant or both said donor and recipient plants produce a recombinase recognizing said recombination sites.

4. The method of claim 1, wherein said donor and recipient plants are crossed sexually.

5. The method of claim 1, wherein said donor and recipient plants are crossed by somatic cell fusion.

6. The method of claim 1, wherein the donor plant and the recipient plant belong to the same taxonomic family but to different species or subspecies.

7. The method of claim 1, wherein the donor plant and the recipient plants belong to the same genus but to different species or subspecies.

8. The method of claim 5, wherein said donor and recipient plants belong to different taxonomic families.

9. The method of claim 1, wherein said donor plant is Tripsacum and said recipient plant is barley.

10. The method of claim 1, wherein said donor plant is Tripsacum and said recipient plant is oat.

11. The method of claim 1, wherein said donor plant is Orychophragmus and said recipient plant is a crucifer.

12. The method of claim 11, wherein said crucifer is canola or rape seed.

13. The method of claim 1, wherein said donor plant is Glycinetomentella and said recipient plant is soybean.

14. The method of claim 1, wherein said donor plant is Solanum phreja and said recipient plant is potato.

15. The method of claim 1, wherein said donor plant is maize and said recipient plant is wheat.

16. The method of claim 1, wherein said donor plant is maize and said recipient plant is barley.

17. The method of claim 1, wherein said donor plant is maize and said recipient plant is oats.

18. The method of claim 1, wherein said donor plant is Pennisetum and said recipient plant is wheat.

19. The method of claim 1, wherein said donor plant is Pennisetum and said recipient plant is barley.

20. The method of claim 1, wherein said donor plant is Pennisetum and said recipient plant is maize.

21. The method of claim 1, wherein said donor plant is Hordeum bulbosum and said recipient plant is barley.

22. The method of claim 1, wherein said donor plant is Hordeum bulbosum and said recipient plant is wheat.

23. The method of claim 1, wherein said donor plant is Oryza minuta and said recipient plant is rice.

24. The method of claim 1, wherein said donor plant is Tripsacum and said recipient plant is wheat.

25. The method of claim 1, wherein said donor plant is Tripsacum and said recipient plant is maize.

26. The method of claim 1, wherein said donor or said recipient plant is soybean carrying a ms mutation causing polyembryony.

27. The method of claim 1, wherein one or both of said donor and recipient plants is cotton carrying a Se semigamy mutation.

28. The method of claim 1, wherein said donor plant is Nicotiana africana and said recipient plant is Nicotiana tabacum.

29. The method of claim 1, wherein said nucleic acids having sequences that allow for site-specific recombination additionally comprise fragments of a selectable marker gene, whereby the selectable marker becomes functional as a result of legitimate site-specific recombination.

30. The method of claim 1, wherein said nucleic acids having sequences that allow for site-specific recombination additionally comprise transposon element(s), whereby the transposon element(s) can be used for mapping resultant chromosome recombinants by insertional mutagenesis.

31. A library of chromosomal fragments of one or more heterologous species incorporated into a genome of a crop species, obtained by the method of claim 1.

32. Breeding material of crop species obtained by the method of claim 1.

33. Plants or plant material obtained by the method of claim 1 and products derived therefrom.
